(19) United States
(12) Patent Application Publication
(10) Pub. No.: US 2025/0136967 A1
(43) Pub. Date: May 1, 2025

(54) REME-D NUCLEIC ACID EXTRACTION KITS

(71) Applicant: REME-D, INC., Cairo (EG)

(72) Inventors: Salma N. TAMMAM, Cairo (EG); Mariam A. MONTASER, Cairo (EG); Esraa E. SAYED, Cairo (EG)

(73) Assignee: REME-D, INC., Cairo (EG)

(21) Appl. No.: 18/498,571

(22) Filed: Oct. 31, 2023

(51) Int. Cl. C12N 15/10

(52) U.S. Cl. CPC C12N 15/1013

(57) ABSTRACT

The invention pertains to methods and kits that simplify and speed the purification and recovery of RNA and DNA and avoid the use of lytic and digestive agents such as proteinase K thus avoiding. These kits are stable at room temperature and reduce the costs and processing time for isolation purification of RNA or DNA.

### BACKGROUND OF THE INVENTION

Field of the invention The invention relates to methods and kits for rapid isolation of nucleic acids from a sample.

Description of related art Methods for affinity purification of nucleic acids are known and are embodied in various commercially available kits such as those available from QIAamp DNA Mini Kit by Qiagen (<https://www.pcr-lab-products.com/genomic-dna-purification/?language=enqiagen>, incorporated by reference) and GeneAll silica columns (South Korea; <https://www.pcr-lab-products.com/genomic-dna-purification/?language=en>, incorporated by reference).

Kits employing magnetic particles that bind to nucleic acids that can be magnetically separated from unbound components of a sample are also commercially available. These include kits produced by Abbott or kits that employ GeneAll magnetic beads (South Korea). Existing kits and methods involve multiple steps and complex buffers. These are undesirable when conducting urgent experiments, real-time protocols, time-sensitive assays, high-throughput workflows, forensic analysis, or critical clinical diagnoses. Moreover, longer and more complex protocols increase the risk of degradation of a nucleic acid sample by nucleases, enzymes, and other chemical mechanisms during processing.

Prior attempts to eliminate Proteinase K from DNA/RNA extraction processes include liquid-based extraction processes described by of Lahiri, et al., A non-organic and non-enzymatic extraction method gives higher yields of genomic DNA from whole-blood samples than do nine other methods tested. J. BIOCHEM BIOPHYS METHODS. 1992 December; 25 (4): 193-205. doi: 10.1016/0165-022x(92)90014-2. PMID: 1494032 (Table A, below). However, these methods are lengthy and not suitable for high-throughput applications, see Han P, et al. A high-throughput pipeline for scalable kit-free RNA extraction. SCI REP. 2021 Dec. 1; 11 (1): 23260. doi: 10.1038/s41598-021-02742-w. PMID: 34853385; PMCID: PMC8636496. Moreover, most of these methods with the exception of protocols 1, 2 and 10 in Table A mandate the use of phenol and chloroform which are toxic and can contaminate a sample.

TABLE A
Brief classification of different methods for extracting DNA from blood

| Steps/Methods | 1 | 2 | 3 | 4 | 5 | 6 | 7 | 8 | 9 | 10a | 10b |
|---|---|---|---|---|---|---|---|---|---|---|---|
| Leukocyte separation | | | | | | | | | | | |
| PBS | + | − | − | + | − | − | − | − | − | − | − |
| Gradient (HP-1077) | − | + | − | − | − | − | − | + | − | − | − |
| Trition X-100 | − | − | + | − | − | + | − | − | − | − | + |
| NP-40 | − | − | − | − | + | − | − | − | + | + | − |
| High Tris/NH₄Cl | − | − | − | − | − | − | + | − | − | − | − |
| Nuclear lysis | | | | | | | | | | | |
| HS/SDS * | + | ? | − | + | − | − | − | + | + | + | + |
| MS/EDTA/SDS ** | − | − | + | − | + | + | − | − | − | − | − |
| High Tris/EDTA | − | − | − | − | − | − | + | − | − | − | − |
| Deproteinization | | | | | | | | | | | |
| Proteinase K/SDS | + | + | − | + | + | + | + | + | − | − | − |
| Saturated NaCl | + | − | − | − | − | − | − | − | − | + | + |
| Chloroform | − | − | + | + | + | + | + | + | + | − | − |
| Phenol | − | − | − | + | + | + | + | + | + | − | − |
| RNase A treatment | − | + | + | − | − | − | − | − | − | − | − |
| DNA precipitation | + | + | + | + | + | + | + | + | + | + | + |
| Time of extraction | O/N | 5 h | 3 h | O/N | O/N | 6 h | 6 h | O/N | 3 h | 1 h | 1 h |
| A₂₆₀/A₂₈₀ | 1.75 | 1.70 | 1.81 | 1.72 | 1.72 | 1.94 | 1.81 | 1.74 | 1.70 | 1.81 | 1.8 |
| Yield of DNA (μg) | 116 | 95 | 135 | 55 | 75 | 174 | 147 | 130 | 170 | 210 | 175 |
| (S.E. − S.D. + \sqrt{n}) | ±31 | ±5 | ±9 | ±2 | ±14 | ±27 | ±12 | ±19 | ±10 | ±28 | ±26 |

Flowsheet diagram of various steps involved in different methods of DNA extractions (Methods 1-10b), as described in the text. steps used in a method are shown under that method as ‘+’. Abbreviations used: * HS/SDS and ** MS/SDS represent a group of buffers that contain high salt (HS) or medium salt (MS) and SDS, whose exact concentration might vary among the different methods as described in the text; O/N, overnight; S.E., standard error; S.D., standard devation.

Table A describes extraction methods 1 to 10b and reports the time of extraction, ranging from 1 hr to overnight, and the A260/A280 ratios of extracted DNA which indicate the degree of purity and which range from 1.7 to 1.81. The ratio of absorbance at 260 nm and 280 nm is used to assess the purity of DNA and RNA. A ratio of 1.8-2.0 indicated pure nucleic acids. If the ratio is low it indicates the presence of protein, phenol or other contaminants that absorb strongly at or near 280 nm. When considering the proteinase K, phenol, chloroform free methods (1, 2 and 10a and b), methods 1 and 2 show a low A260/A280 ratio and only 10Aa and b show values that fall within the acceptable range. These methods however are still rather lengthy.

In distinction to the methods described in Table A, when MG-NA and COL-NA kits and methods were used to isolates nucleic acids from whole blood the A260/A280 ratio for COL-NA was 2.01 whereas for MG-NA it was 1.9.

Considering that shorter and less complicated protocols could reduce the amount of skilled labor required and costs for nucleic acid purification, the inventors sought to develop a nucleic acid purification method and kit that was effective, simple, easy to use, and more rapid than existing methods.

### BRIEF SUMMARY OF THE INVENTION

One aspect of the invention is a rapid and efficient spin column-based method for extracting and purifying nucleic acids including DNA or RNA from a variety of different samples. This method is designated COL-NA. As shown below this method efficiently isolates nucleic acids from a sample but is less complex and more rapid than existing spin column-based methods.

Another aspect of the invention is a rapid and efficient magnetic particle based method for extracting and purifying nucleic acids, including DNA or RNA, from a sample. This method is designated MG-NA. As shown below this method efficiently isolates nucleic acids from a variety of biological samples but is faster and less complex than existing magnetic particle based methods.

A further aspect of the invention is a COL-NA or MG-NA kit useful for performing the COL-NA and MG-NA methods. Typically, a kit contains a nucleic acid binding material, lysis buffers, nucleic acid binding buffers, wash buffers, and elution buffers.

### BRIEF DESCRIPTION OF THE DRAWINGS

### COL-NA Figures

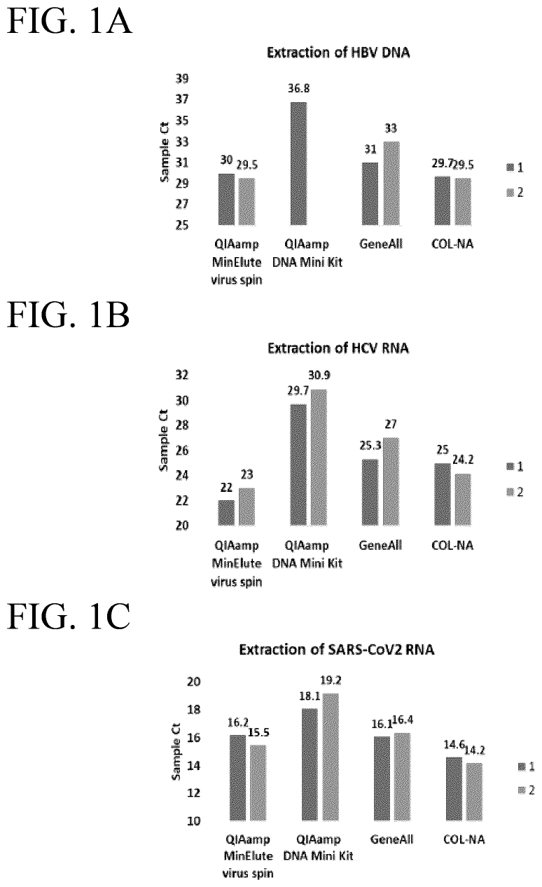

FIGS. 1A-1C. Compare the performance of COL-NA to commercially available kits. The same sample was analyzed twice by each of the kits.

Samples from HBV positive and SARS-CoV2 positive patients were extracted by COL-NA and by the commercially available kits. Subsequently, real-quantitative polymerase chain reaction (RT-qPCR) was performed. As shown, the COL-NA kits bad the highest ability to extract HBV DNA and SARS-CoV2 RNA as indicated by the lowest Ct values. When samples from HCV positive patients were extracted by the aforementioned kits followed by real-quantitative polymerase chain reaction (RT-qPCR), QIAamp MiniElute Virus Spin kit yielded slightly lower Cts in comparison to COL-NA. COL-NA however showed superior performance in comparison to QIAamp DNA Mini Kit by Qiagen and GeneAll, silica columns (South Korea).

FIG. 1A. Extraction of HBV DNA using (a) QIAamp MiniElute Virus Spin kit, (b) QIAamp DNA Mini Kit by Qiagen and (c) GeneAll silica columns (South Korea) and (d) COL-NA (invention).

FIG. 1B. Extraction of HCV RNA using (a) QIAamp MiniElute Virus Spin kit, (b) QIAamp DNA Mini Kit by Qiagen and GeneAll, (c) silica columns (South Korea) and (d) COL-NA (invention).

FIG. 1C. Extraction of SARS-COV-2 RNA using (a) QIAamp MiniElute Virus Spin kit (Cat. No./ID 57704, (b) QIAamp DNA Mini Kit by Qiagen (Cat., Nos. 51304 and 51306) and (c) GeneAll (EXGENE™ ViralDNA/RNA kit;<https://geneall.com/en/sub/products/prod.asp?mode=view&idx=329&s_keyword=&s_cate=24&s_filter=&s_browser=&s_display=&s_align=0 >) silica columns (South Korea), and (d) COL-NA (invention).

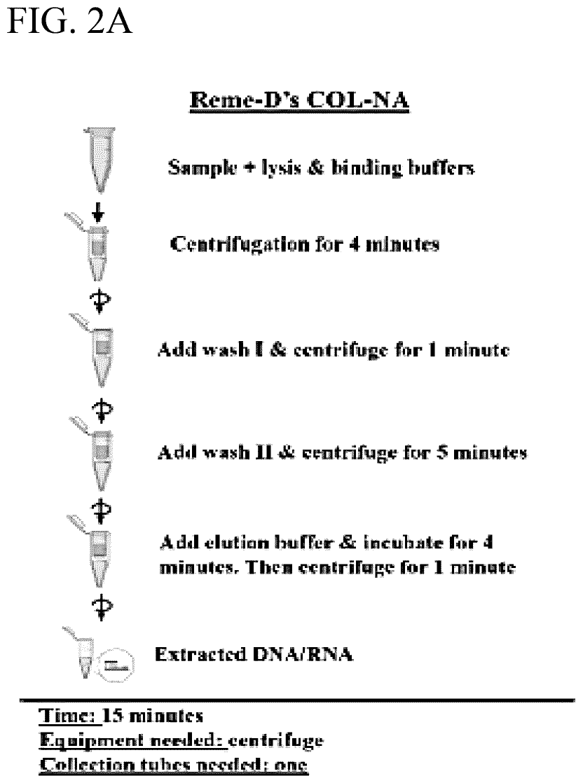

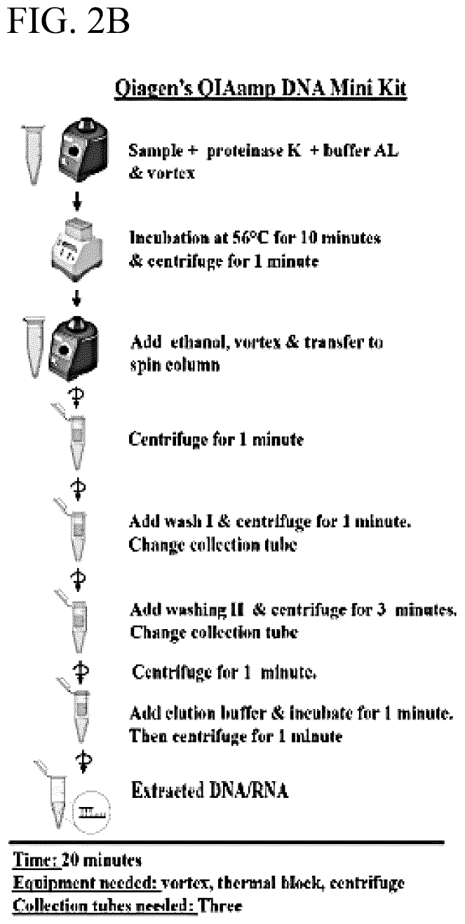

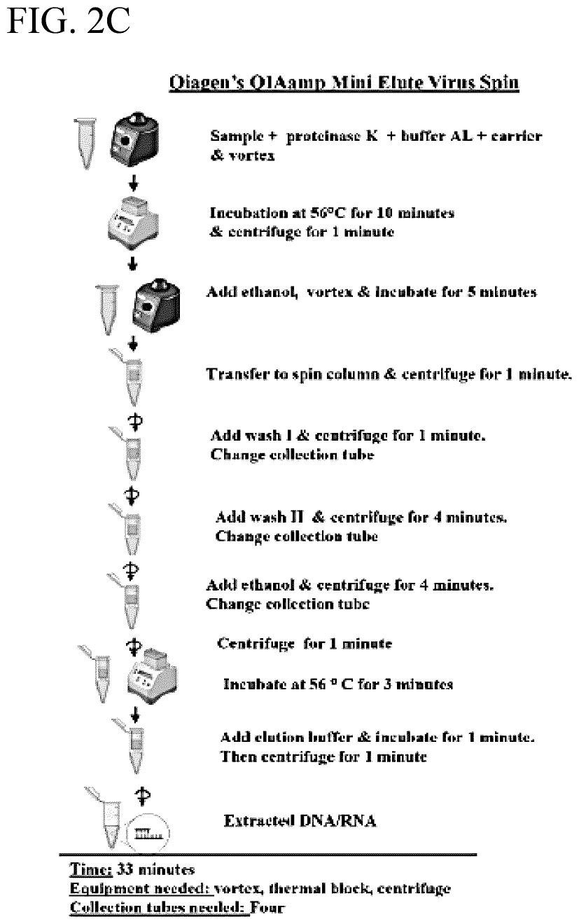

FIGS. 2A-2C. Comparison of extraction protocols (FIG. 2A) COL-NA to (FIG. 2B) QIAamp DNA Mini Kit by Qiagen and to (FIG. 2C) QIAamp MiniElute Virus Spin kit. As apparent from the parallel diagrams, the COL-NA extraction protocol is simpler and took less time than those of the two conventional nucleic acid extraction kits.

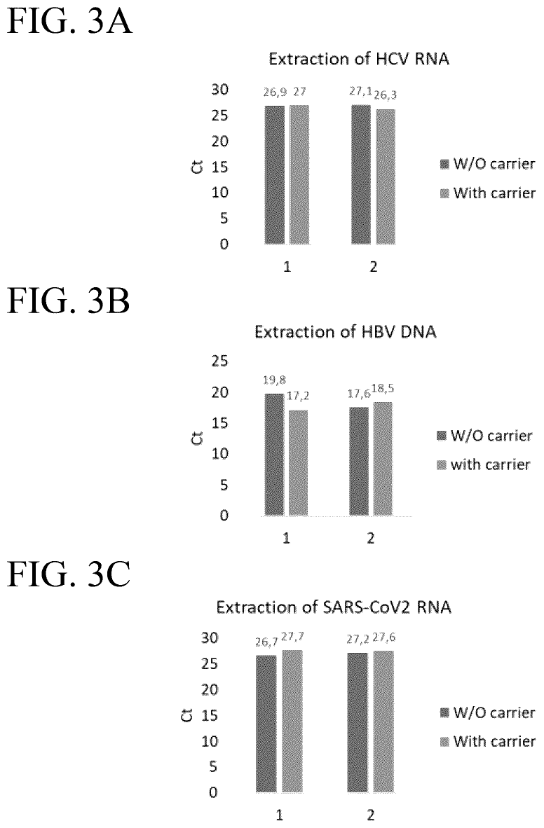

FIGS. 3A-3C. Effect of a Qiagen carrier (Poly-A) on extraction efficiency of the COL-NA kit of the invention for HCV RNA, HBV DNA and SARS-COV-2 RNA. As shown extraction efficiency is similar whether or not a carrier is used. The Qiagen carrier is a polynucleotide “Poly-A” type carrier. It is commercially available from Qiagen as part of a kit or as Poly-A Carrier RNA per se without any buffers under catalog numbers: 1017647: 12 vials, each containing 1350 μg lyophilized carrier RNA and 1068337:1 vial containing 310 μg lyophilized carrier RNA. Results were obtained from COL-NA in comparison to COL-NA+the Poly-A Carrier from Qiagen. Tests were performed in duplicate, thus in FIG. 3A sample 1 and sample 2 are duplicates.

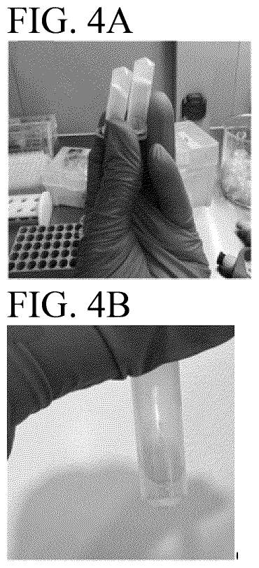

FIGS. 4A and 4B show the effects of including Proteinase K in a lysis buffer of the conventional Qiagen commercial extraction kits. In the Qiagen kit, the serum samples turn into a gel when proteinase-K-free lysis buffer is added (FIG. 4A) but remain in a liquid state when the Reme-D Proteinase K-free lysis buffer according to the invention is added (FIG. 4B). In addition to avoiding this gelling effect, the omission of proteinase K eliminated the need to refrigerate the proteinase K, reduced costs as proteinase K is expensive, avoided the need to incubate the sample with proteinase K (e.g. a 10 min incubation for proteinase K to work). In FIG. 4A both tubes contained serum samples that were processed using Qiagen lysis buffer without the addition of Proteinase K as provided in the Qiagen kit. In FIG. 4B, the same serum sample was processed with the Proteinase K-free lysis buffer of the invention disclosed herein.

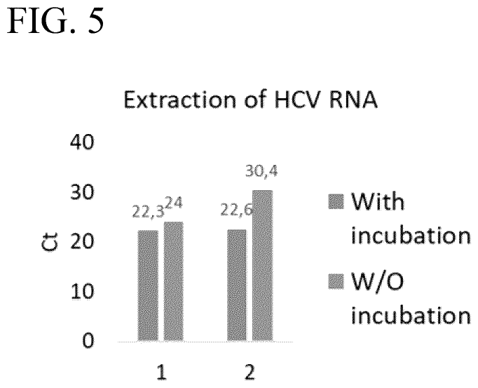

FIG. 5 describes the extraction efficiency of HCV RNA when the 10 min incubation step required by the conventional Qiagen kits is omitted. As shown, the efficiency of extraction is reduced when the incubation step is omitted. The COL-NA method of the invention does not require an incubation step.

The same HCV-positive serum sample was run twice using the Qiagen kits with the recommended incubation described in the kit's protocol and also without it. It is apparent from the results shown that the use of the Qiagen kit without incubation does not provide a reproducible result-see the difference between the second bar in sample 1 (Ct: 24) and the second bar in sample 2 (Ct: 30.4). In contrast the samples that were incubated had a similar yield (Ct: 22.3 and 22.6). This shows the criticality of the 10 min incubation step for the Qiagen kits which is a step not required by COL-NA.

### MG-NA Figures

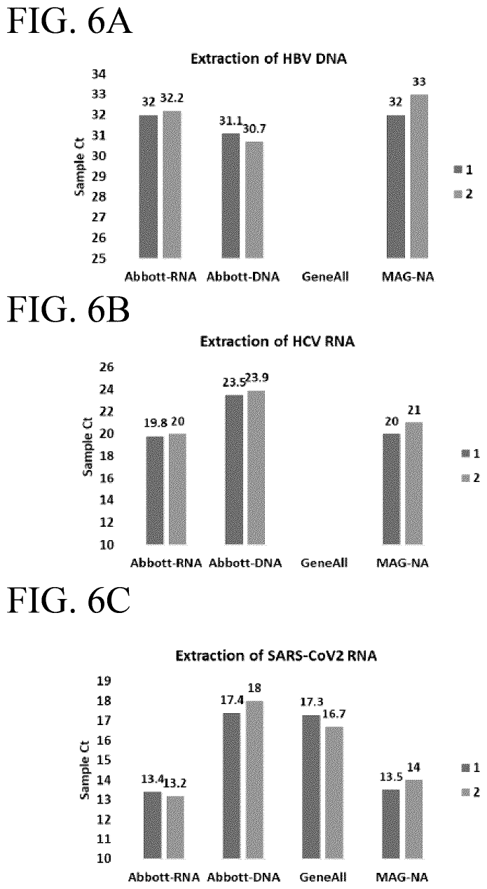

FIGS. 6A-6C show the performance of MG-NA according to the invention in comparison to other commercial kits that also employ magnetic beads: the mSample preparation system (RNA) and mSample preparation system (DNA) by Abbott and GeneAll magnetic beads (South Korea). Further description of these kits is incorporated by reference to mSample preparation system (RNA) Product Number 01N8401; <https://www.e-abbott.com/msample-preparation-system-rna-quick-ref.html>; mSample preparation system (DNA) Product Number 01N8301<https://www.e-abbott.com/msample-preparation-system-dna-quick-ref.html> (each last accessed Aug. 29, 2023).

The same sample was analyzed twice by each of the kits. For all viruses, MG-NA provided results that were similar to those obtained with the kits from Abbott. GeneAll magnetic beads showed mediocre performance when samples from HBV and HCV patients were extracted, a detectable amount of RNA/DNA was not extracted; see FIGS. 6A and 6B.

FIG. 6A. Extraction of HBV DNA using (a) Abbott-RNA kit, (b) Abbott-DNA and (c) GeneAll, silica columns (South Korea) and (d) MAG-NA (invention).

FIG. 6B. Extraction of HCV RNA using (a) QIAamp MiniElute Virus Spin kit, (b) QIAamp DNA Mini Kit by Qiagen and (c) GeneAll silica columns (South Korea) and (d) COL-NA (invention).

FIG. 6C. Extraction of SARS-COV-2 RNA using (a) QIAamp MiniElute Virus Spin kit, (b) QIAamp DNA Mini Kit by Qiagen and (c) GeneAll silica columns (South Korea) and (d) COL-NA (invention). The following commercial products were used for the procedures described above and each is incorporated by reference (each last accessed Aug. 31, 2023).

QIAamp MiniElute Virus Spin kit Cat. No./ID: 57704; <https://www.qiagen.com/us/products/discovery-and-translational-research/dna-rna-purification/multianalyte-and-virus/qiaamp-minelute-virus-kits>.

QIAamp DNA Mini Kit Cat. No./ID: 51304; <https://www.qiagen.com/us/products/discovery-and-translational-research/dna-rna-purification/dna-purification/genomic-dna/qiaamp-dna-kits?catno=51304>.

GeneAll Kit is the EXGENE™ Viral DNA/RNA kit (A cat number is not available) <https://geneall.com/en/sub/products/prod.asp?mode=view&idx=329&s_keyword=&s_cate=24 &s_filter=&s_browser=&s_display=&s_align=0>.

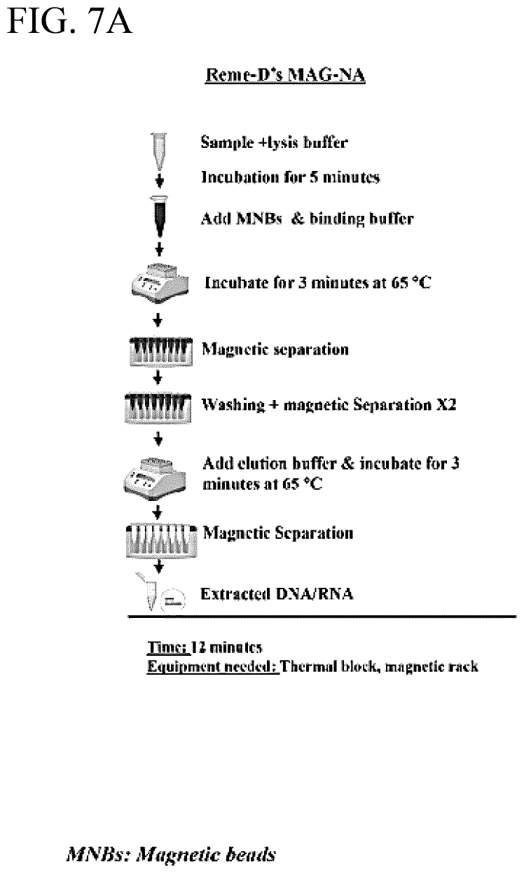

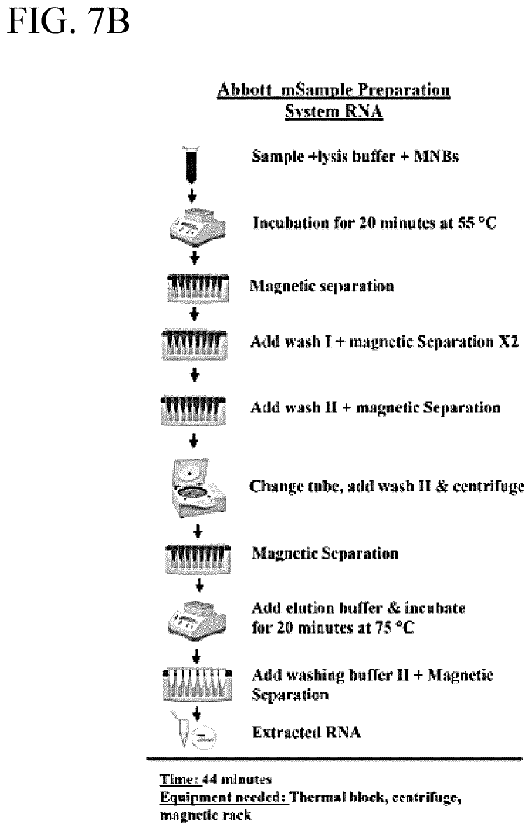

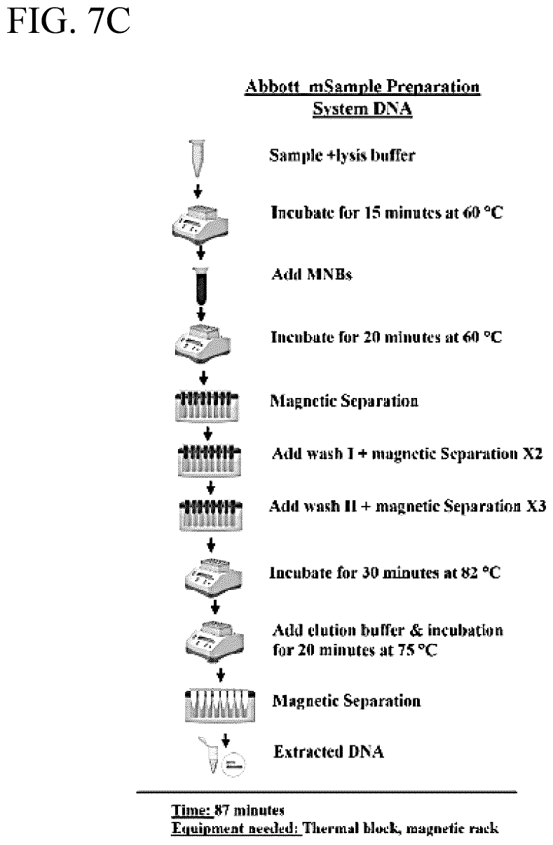

FIG. 7A-7C compare the MG-NA extraction protocol (invention) to the Abbott RNA and Abbott DNA extraction protocols. As apparent from the parallel process flow diagrams, the MG-NA extraction protocol is simpler and takes less time than those of the conventional nucleic acid extraction kits.

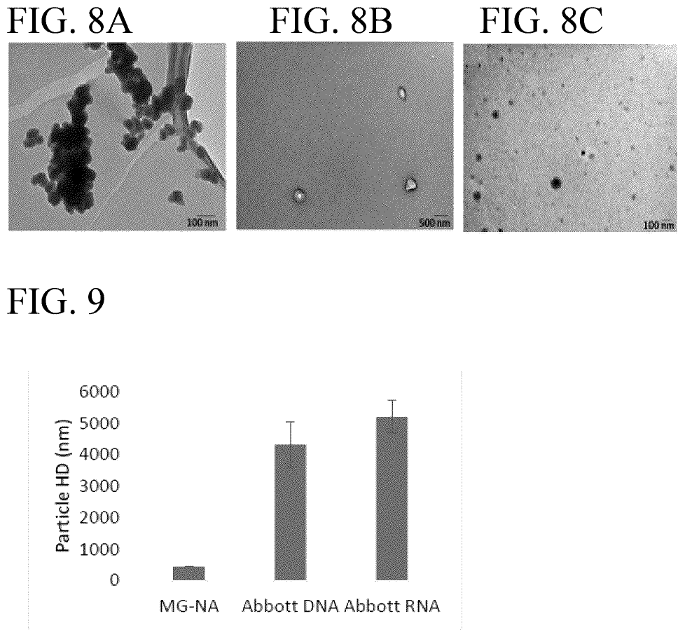

FIGS. 8A-8C. Transmission Electron Microscopy Images of magnetic beads used in MG-NA. TEM images in FIGS. 8A-8C show that the core particle size of particles used in MG-NA is a lot smaller. Within the sample matrix due to the high salt concentrations and the presence, particle aggregation also occurs.

FIG. 8A. Transmission Electron Microscopy Images of magnetic beads used in MG-NA.

FIG. 8B. Transmission Electron Microscopy Images of magnetic beads used in mSample preparation system (DNA) by Abbott.

FIG. 8C. Transmission Electron Microscopy Images of magnetic beads used in m Sample preparation system (RNA) by Abbott. These images were generated for the beads prior to their addition to the sample matrix.

FIG. 9 shows the particle hydrodynamic diameter (HD) when suspended in the lysis/binding buffer. It also shows that the HD of particle used in MG-NA was the smallest and thus had the largest surface area to volume ratio during the extraction reaction.

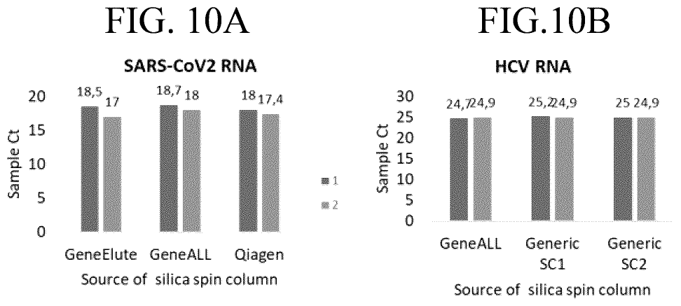

FIGS. 10A and 10B respectively compare recovery of SARS-CoV2 RNA and HCV RNA recovery, when different commercially available silica spin columns were used. Tests were performed in duplicate. The similar Ct values show that nucleic acid recovery did not depend on the source of the spin column.

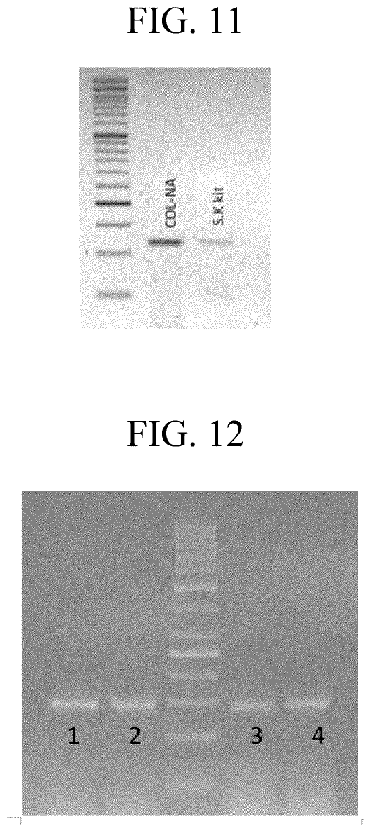

FIG. 11. A compares the ability of COL-NA and the commercial kit (GeneAll) to extract and recover COL3 mRNA from mouse liver. As apparent from the heavier band in lane 2, the COL-NA kit recovered a higher amount of nucleic acid.

FIG. 12. Four replicas resolved using gel electrophoresis of amplified COL3 mRNA that was purified from mouse liver homogenate using MG-NA.

### DETAILED DESCRIPTION OF THE INVENTION

Among its other aspects and embodiments, the invention is directed to two nucleic acid extraction kits: COL-NA & MG-NA and to methods for recovering nucleic acids using them. The COL-NA kit and method employ silica spin columns for the isolation of nucleic acids while MG-NA kit and method employ magnetic nanobeads that bind to nucleic acids. As shown herein both viral DNA and viral RNA as well as nucleic acids from animal sources (liver homogenates) are recoverable by the COL-NA and MG-NA methods and kits. Moreover, the recovery process is simpler and faster than conventional DNA extraction kits that are commercially available such as those made by Qiagen and Abbott. Specific embodiments include but are not limited to the following.

One aspect of the invention is directed to a nucleic acid extraction kit comprising a solid-phase matrix such as silica particles, silica gel, or anion exchange resins that bind nucleic acid such as DNA or RNA or modified DNA or RNA, at least one lysis buffer, binding buffer, wash buffer, and elution buffer. In one embodiment, the kit comprises a single lysis buffer, a single binding buffer, two wash buffers, and a single elution buffer.

In some preferred embodiments of COL-NA kits, the solid phase matrix is contained in a commercially available spin column such as those available from ECONOSPIN®, PUROSPIN™ and QIAGEN®. These columns generally bind to nucleic acid sequences but in some embodiments may be functionalized to bind to specific nucleic acid sequences. In some embodiments, the spin column is composed of four, five or six layers of SiO₂ with an average thickness of 1-3 μm each.

In other embodiments suitable for MG-NA, a spin column is not used and the nucleic acids in the sample bind to a solid phase matrix like silica coated magnetic beads.

Typically a nucleic acid sequence will have a negatively charged backbone which binds with high affinity to positive charges on a silica matrix. In preferred embodiments, binding occurs under chaotropic conditions in the presence of guanidium thiocyanate.

In some embodiments, the kits in addition to a spin column or magnetic beads, and the lysis, binding, wash and elution buffers further comprise a collection rack and/or at least one collection tube or containers for the separate buffers or for samples. They may also contain labels, packaging materials, or instructions.

In some embodiments suitable for COL-NA, the nucleic acid extraction kit comprises lysis buffer comprises Tris-HCl (pH 7), NaCl, guanidine thiocyanate, Na-EDTA, SDS and a buffer that adjusts a pH suitable for lysis or digestion of the sample to release nucleic acids. In other embodiments, the nucleic acid extraction kit contains a lysis buffer comprising 30 mM Tris-HCl (pH 7), 1M NaCl, 4.5M guanidine thiocyanate (or a chaotropic amount), 20 mM Na-EDTA, 0.5% w/v SDS at a final buffer pH of 5.5. However, the concentrations of each buffer may vary by +1, 2, 5, 10, 15 or 20%, and the pH may vary from 5.0, 5.1, 5.2, 5.3, 5.4, 5.5, 5.6, 5.7, 5.8, to 6.0. In a related embodiment suitable for COL-NA, the invention comprises a lysis buffer suitable for COL-NA or a kit or assembly of lysis, binding, wash and/or elution buffers.

In embodiments suitable for MG-NA, the lysis buffers described above which are suitable for COL-NA, further comprise β-mercaptoethanol, for example, at a concentration of about 0.01, 0.02, 0.03, 0.04, 0.05, 0.06, 0.07, 0.08, 0.09, 0.1, 0.2, 0.3, 0.4, 0.5 or >0.5 v/v % or at a concentration effective for reduction of disulfide bonds in a biological sample. For example, a suitable MG-NA buffer may comprise 30 mM Tris-HCl (pH 7), 1M NaCl, 4.5M guanidine thiocyanate, 20 mM Na-EDTA, 0.5% w/v SDS, and 0.1 v/v % β-mercaptoethanol at a final pH of 5.5, wherein the concentration of each ingredient may vary by #1, 2, 5, 10, 15 or 20%, and wherein the pH may vary from 5.0, 5.1, 5.2, 5.3, 5.4, 5.5, 5.6, 5.7, 5.8, to 6.0. Other reductants such as DTT, TCEP or 2-mercaptoethanol may be substituted for β-mercaptoethanol or added into the MG-NA lysis buffer in amounts sufficient to reduce disulfide bonds in proteins or other contaminants. In a related embodiment suitable for MG-NA, the invention comprises a lysis buffer suitable for MG-NA or a kit or assembly of lysis, binding, wash and/or elution buffers.

In some embodiments suitable for either COL-NA or MG-NA the nucleic acid extraction kit contains a binding buffer which comprises an alcohol such as 60, 65, 70, 80, 90, 95, 96, 97, 98, 99 or 100% (or any intermediate concentration) isopropyl alcohol, preferably about 100% isopropanol. Other alcohols or chaotropic agents that may be used in a binding buffer comprise n-butanol, ethanol, 2-propanol, lithium perchlorate, lithium acetate, magnesium chloride, phenol, SDS, thiourea, urea or other chaotropic agents. These are incorporated at a strength or amount sufficient to facilitate or stabilize nucleic acid binding to the matrix.

The kit may contain one, two, three or more wash buffers formulated to not detach nucleic acids from the solid phase matrix but remove contaminating molecules. In preferred embodiments, the wash buffer comprises a first and second wash buffer, wherein the first wash buffer, which is suitable for COL-NA or MG-NA comprises: Tris-HCl, guanidine thiocyanate, ethanol; and wherein the second wash buffer suitable for COL-NA comprises Tris-HCl (pH 7) and ethanol; or comprises a diluted NaOH solution at pH 10.8 suitable for MG-NA.

In another preferred embodiment, the wash buffers comprise a first and second wash buffer, wherein the first wash buffer comprises 30 mM Tris-HCl (about pH 7), 1.125 M guanidine thiocyanate, 56% v/v ethanol and a buffer that adjusts pH to about 7.2 wherein the concentration of each ingredient in the first wash buffer may vary by +1, 2, 5, 10, 15 or 20%, and wherein the pH of the first wash buffer may vary from 6.13 to <9; and wherein the second wash buffer comprises for COL-NA 10 mM Tris-HCl (about pH 7) in 70% v/v ethanol and a buffer that adjusts pH to about 6.3 to 8.3; and the second wash buffer for MG-NA has an alkaline pH up to pH 11, preferably about pH 10.8.

In some embodiments, the nucleic acid extraction kit comprises an elution buffer that is water, a low salt buffer containing 10-50 mM monovalent salt ions, or a commercially available nucleic acid elution buffer. Preferably the elution buffer is deionized water.

In some embodiments, the nucleic extraction kit further comprises a carrier such as linear polyacrylamide, glycogen, tRNA, sodium acetate, or another carrier or nucleic acid precipitant to enhance nucleic acid binding to the solid phase matrix. In other preferred embodiments, the kit does not contain a carrier.

In some embodiments, the nucleic acid extraction kit further comprises a filter or other device suitable for separating the nucleic acid bound to the solid-phase matrix from unbound fluids and materials, containers for nucleic acid containing samples or for buffers, packaging materials, labels, and/or a user manual or instructions for use.

Another aspect of the invention is directed to method for extracting a nucleic acid, such as DNA, RNA, amplified DNA or RNA, or modified DNA or RNA from a sample comprising contacting the sample with a solid-phase matrix that binds nucleic acid and with a lysis buffer for a time and under conditions suitable for release of the nucleic acid from other components in the sample, contacting the released nucleic acid with a binding buffer under conditions suitable for binding or enhanced binding of the nucleic acid to the solid phase matrix, separating the nucleic acid bound to the solid-phase matrix from the unbound portion of the sample, washing the separated nucleic acid bound to the solid-phase matrix with a wash buffer to remove unbound components of the sample, optionally repeating the washing step one, two, three or more times with the same or a different wash buffer, and eluting the nucleic acid bound to the solid-phase matrix from the solid phase matrix by contacting it with an elution buffer. The buffers described herein for the extraction method may be the same as those described above for the extraction kits.

One embodiment of this method designated COL-NA involves the use of a spin column containing the solid nucleic acid binding matrix It is directed to a method for extracting a nucleic acid from a sample comprising:
contacting the sample with a lysis buffer for a time and under conditions suitable to release the nucleic acid from other components in the sample,
contacting the released nucleic acid with a binding buffer to facilitate binding of the nucleic acid with a solid phase matrix in a spin column,
passing the nucleic acid through the spin column for a time and under conditions suitable for binding of the released nucleic acid to the solid phase matrix,
washing the solid phase matrix that is bound to the released nucleic acid one or more times to remove unbound components in the sample,
contacting the washed solid phase matrix bound to the nucleic acid with an elution buffer under conditions suitable for release of the bound nucleic acid from the solid phase matrix, thereby extracting the nucleic acid from the sample.

Another embodiment of this method designated MG-NA involves the use of magnetic nanoparticles or magnetic beads bound to a nucleic acid binding matrix. It is directed to a method for extracting a nucleic acid from a sample comprising:
contacting the sample with a lysis buffer for a time and under conditions suitable to release the nucleic acid from other components in the sample,
contacting the released nucleic acid with a binding buffer to facilitate binding of the nucleic acid with a solid phase matrix that binds to nucleic acids bound to magnetic nanoparticles.
contacting the nucleic acid with the magnetic nanoparticles for a time and under conditions suitable for binding of the nucleic acid to the solid phase matrix bound to magnetic nanoparticles,
magnetically separating the nucleic acid bound to the solid phase matrix bound to magnetic nanoparticles,
washing the solid phase matrix bound to magnetic nanoparticles which is bound to the released nucleic acid one or more times to remove unbound components in the sample,
contacting the solid phase matrix bound to magnetic nanoparticles which is bound to the released nucleic acid with an elution buffer under conditions suitable for release of the bound nucleic acid from the solid phase matrix, thereby extracting the nucleic acid from the sample.

In some embodiments, the sample is tissue or tissue homogenate, cells, blood, plasma, serum, tissue fluid, CSF, bronchial fluid, mucous, semen, vaginal fluid, cultured cells, disrupted cells or tissues, or amplified nucleic acids, for example, PCR amplified nucleic acids containing enzymes, proteins or other contaminants. Any biological sample that can be lysed or digested so as to release nucleic acids in recoverable form may be used including samples of bacteria, fungi, animal or plant cells, cerebrospinal fluid, mucosal material, material from nasopharyngeal swabs or biopsy.

In other embodiments, the solid-phase matrix that binds to a nucleic acid is contained in a spin column and the nucleic acid bound to the solid-phase matrix is separated from unbound components of the digested sample by centrifugation. In some embodiments of this method, the eluted nucleic acid is collected in a collection tube.

In some embodiments of this method the solid-phase matrix comprises silica particles or other nucleic acid binding particles or a silica gel membrane.

In other embodiments, the solid-phase matrix will comprise magnetic beads or particles suitable for use in MG-NA. For example, the matrix may comprise magnetic nanobeads that have a core size of no more than 20, 25, 30, 35, 40, 45, 50, 100, 200, 300, 400 or 500 nm by transmission electron microscopy bound to silica beads or other nucleic acid binding particles. HD may be measured or determined by dynamic light scattering (DLS) or nanoparticle tracking analysis (NTA).

In some embodiments of this method the lysis buffer comprises Tris-HCl (pH 7), NaCl, guanidine thiocyanate, Na-EDTA, SDS and a buffer that is adjusted to a pH suitable for lysis or digestion of the sample to release nucleic acids. In preferred embodiments, the lysis buffer preferably contains one or more detergents at concentrations above their critical micelle concentration. In other alternative embodiments, the detergent content can be below the CMC.

In some preferred embodiments the lysis buffer does not contain proteinase K or another protease. In others it does.

In additional embodiments of this method the lysis buffer comprises 30 mM Tris-HCl (pH 7), 1M NaCl, 4.5M guanidine thiocyanate, 20 mM Na-EDTA, 0.5% w/v SDS-Buffer final pH 5.5, wherein the concentration of each ingredient may vary by +1, 2, 5, 10, 15 or 20%, and wherein the pH may vary from 5.0, 5.1, 5.2, 5.3, 5.4, 5.5, 5.6, 5.7, 5.8, to 6.0. The lysis buffer suitable for MG-NA may further comprise β-mercaptoethanol or another reductant.

In some embodiments the binding buffer which comprises an alcohol such as 60, 70, 80, 90-100% isopropyl alcohol, preferably about 100% isopropanol. Other alcohols or chaotropic agents that may be used in a binding buffer comprise n-butanol, ethanol, 2-propanol, lithium perchlorate, lithium acetate, magnesium chloride, phenol, SDS, thiourea, urea or other chaotropic agents.

In some embodiments, the binding buffer does not contain detergent.

In some embodiments of this method, the wash buffer comprises a first and second wash buffer, wherein the first wash buffer comprises: Tris-HCl, guanidine thiocyanate, ethanol; and wherein the second wash buffer comprises Tris-HCl (about pH 7) and ethanol for COL-NA, or for MG-NA dilute NaOH, and wherein the first and second wash buffers are formulated so that they do not substantially remove nucleic acids from the solid-phase matrix.

In other embodiments of this method, the wash buffer comprises a first and second wash buffer, wherein the first wash buffer comprises 30 mM Tris-HCl (about pH 7), 1.125 M guanidine thiocyanate, 56% v/v ethanol and a buffer that adjusts pH to about 7.2 wherein the concentration of each ingredient in the first wash buffer may vary by +1, 2, 5, 10, 15 or 20%, and wherein the pH of the first wash buffer may vary from 6.13 to <9; and wherein the second wash buffer comprises 10 mM Tris-HCl (about pH 7) in 70% v/v ethanol and a buffer that adjusts pH to about 6.3 to 8.3 for COL-NA and wash buffer 2 for MG-NA at a pH range up to pH 10, 10.1, 10.2, 10.3, 10.4, 10.5, 10.6, 10.7, 10.8, 10.9 or 11, preferably about pH 10.8 for MG-NA wash buffer 2.

In some embodiments of this method, the elution buffer is water, a low salt buffer containing 10-50 mM monovalent salt ions, or a commercially available nucleic acid elution buffer and is preferably, pure deionized water.

In some embodiments of this method, the lysis, binding buffer, or wash buffers further comprise a carrier such as linear polyacrylamide, glycogen, tRNA, sodium acetate, or another carrier or nucleic acid precipitant to enhance nucleic acid binding to the solid phase matrix. In other preferred embodiments, the method does not use a carrier.

In some embodiments, the sample comprises DNA or modified DNA. In some embodiments, when DNA is recovered by the methods disclosed herein and RNAse is added to one or more of the buffers to eliminate RNA contamination.

In some embodiments, the sample comprises RNA or modified RNA. In some embodiments, when RNA is recovered by the methods disclosed herein an DNAse is added to one or more of the buffers to eliminate DNA contamination.

In other embodiments, the sample comprises RNA and the lysis, binding, wash, and elution buffers do not contain an RNAse inhibitor. In other embodiments, one or more of the buffers used in this method do contain RNAse inhibitors.

In some embodiments, the kits and methods disclosed herein produce eluted nucleic acids such as DNA or RNA that have an A260/A280 ratio ranging from 1.5, 1.6, 1.7, 1.8, 18.5, 1.9, 1.95 to 2.0.

In some embodiments, the kits and methods disclosed herein produce eluted nucleic acids such as DNA or RNA within a time period or extraction time of 10, 11, 12, 13, 14, 15, 16, 17, 18, 19, 20, 21, 22, 23, 24, 25, 26, 27, 28, 29, 30 mins or less.

In some embodiments, the kits and methods disclosed herein produce eluted nucleic acids such as DNA or RNA that have an A260/A280 ratio ranging from 1.5, 1.6, 1.7, 1.8, 18.5, 1.9, 1.95 to 2.0 within a time period of (or total extraction time of) 10, 11, 12, 13, 14, 15, 16, 17, 18, 19, 20, 21, 22, 23, 24, 25, 26, 27, 28, 29, 30 mins or less.

Some specific embodiments of the invention include, but are not limited to:
1. A nucleic acid extraction kit comprising a solid-phase matrix that binds nucleic acid, a lysis buffer, a binding buffer, a wash buffer, and an elution buffer.
2. The nucleic acid extraction kit of embodiment 1, wherein the solid phase matrix is contained in a spin column.
3. The nucleic acid extraction kit of embodiment 1 or 2, wherein the solid phase matrix comprises silica particles that bind to nucleic acids.
4. The nucleic acid extraction kit of embodiment 1, 2, or 3, wherein the solid phase matrix comprises four to six layers of SiO₂, wherein each layer has a thickness ranging from 1 to 3 μm.
5. The nucleic acid extraction kit of embodiment 1, 2, 3, or 4, wherein the solid phase matrix comprises silica coated magnetic nano-beads.
6. The nucleic acid extraction kit of any one of embodiments 1-5, wherein the solid phase matrix comprises silica coated magnetic nanobeads that have a core size of no more than 30, 40, or 50 nm by transmission electron microscopy.
7. The nucleic acid extraction kit of any one of embodiments 1-6, further comprising a collection rack and/or at least one collection tube.
8. The nucleic acid extraction kit of any one of embodiments 1-7, that is suitable for use in COL-NA, wherein the lysis buffer comprises Tris-HCl (pH 7), NaCl, guanidine thiocyanate, Na-EDTA, and wherein the lysis buffer is adjusted to a pH suitable for lysis or digestion of the sample and release of nucleic acids.
9. The nucleic acid extraction kit of any one of embodiments 1-8 that is suitable for use in COL-NA, wherein the lysis buffer comprises 30 mM Tris-HCl (pH 7), 1M NaCl, 4.5M guanidine thiocyanate, 20 mM Na-EDTA, 0.5% w/v SDS at a final pH of 5.5, wherein the concentration of each ingredient may vary by +1, 2, 5, 10, 15 or 20%, and wherein the pH may vary from 5.0, 5.1, 5.2, 5.3, 5.4, 5.5, 5.6, 5.7, 5.8, to 6.0.
10. The nucleic acid extraction kit of any one of embodiments 1-7 that is suitable for use in MG-NA, wherein the lysis buffer comprises Tris-HCl (pH 7), NaCl, guanidine thiocyanate, Na-EDTA, SDS, β-mercaptoethanol, wherein the lysis buffer is adjusted to a pH suitable for lysis or digestion of the sample and release of nucleic acids.
11. The nucleic acid extraction kit of any one of embodiments 1-7 or 10 that is suitable for use in MG-NA, wherein the lysis buffer comprises 30 mM Tris-HCl (pH 7), 1M NaCl, 4.5M guanidine thiocyanate, 20 mM Na-EDTA, 0.5% w/v SDS, and 0.1 v/v % β-mercaptoethanol at a final pH of 5.5, wherein the concentration of each ingredient may vary by +1, 2, 5, 10, 15 or 20%, and wherein the pH may vary from 5.0, 5.1, 5.2, 5.3, 5.4, 5.5, 5.6, 5.7, 5.8, to 6.0.
12. The nucleic acid extraction kit of any one of embodiments 1-11, wherein the binding buffer comprises an alcohol.
13. The nucleic acid extraction kit of any one of embodiments 1-12 wherein the binding buffer comprises 70, 80, 90-100% isopropyl alcohol.
14. The nucleic acid extraction kit of any one of embodiments 1-13, wherein the wash buffer comprises a first and second wash buffer,
   wherein the first wash buffer comprises: Tris-HCl, guanidine thiocyanate, ethanol; and
   wherein the second wash buffer comprises Tris-HCl (pH 7) and ethanol suitable for COL-NA, or comprises a diluted NaOH solution at pH 10.8 that is suitable for MG-NA, and
   wherein the first and second wash buffers are formulated so that they do not substantially remove nucleic acids from the solid-phase matrix.
15. The nucleic acid extraction kit of any one of embodiments 1-14, wherein the wash buffer comprises a first and second wash buffer,
   wherein the first wash buffer comprises 30 mM Tris-HCl (about pH 7), 1.125 M guanidine thiocyanate, 56% v/v ethanol and a buffer that adjusts pH to about 7.2 wherein the concentration of each ingredient in the first wash buffer may vary by +1, 2, 5, 10, 15 or 20%, and wherein the pH of the first wash buffer may vary from 6.13 to <9; and
   wherein the second wash buffer comprises 10 mM Tris-HCl (about pH 7), 70% v/v ethanol and a buffer that adjusts pH to about 6.3 to 8.3 and is suitable for COL-NA, or the second wash buffer comprises a diluted NaOH solution at pH 10.6 suitable for MG-NA.
16. The nucleic acid extraction kit of any one of embodiments 1-15, wherein the elution buffer is water, a low salt buffer containing 10-50 mm monovalent salt ions, or a commercially available nucleic acid elution buffer.
17. The nucleic acid extraction kit of any one of embodiments 1-16, wherein the elution buffer is deionized water.
18. The nucleic extraction kit of any one of embodiments 1-17 that does not contain linear polyacrylamide, glycogen, tRNA, sodium acetate, or another carrier or nucleic acid precipitant.
19. The nucleic acid extraction kit of any one of embodiments 1-18, further comprising a centrifuge, filter or other device suitable for separating the nucleic acid bound to the solid-phase matrix from unbound fluids and materials, containers for nucleic acid containing samples or for buffers, packaging materials, and/or a user manual or instructions for use.
20. A method for extracting a nucleic acid from a sample comprising:
   contacting the sample with a solid-phase matrix that binds nucleic acid and with a lysis buffer for a time and under conditions suitable digestion or release of the nucleic acid from other components in the sample,
   contacting the digested or released nucleic acid with a binding buffer under conditions suitable for binding of the nucleic acid to the solid phase matrix,
   separating the nucleic acid bound to the solid-phase matrix from the unbound portion of the sample,
   washing the separated nucleic acid bound to the solid-phase matrix with a wash buffer to remove unbound components of the sample, optionally repeating the washing step one or more times with the same or a different wash buffer, and
   eluting the nucleic acid bound to the solid-phase matrix from the solid phase matrix by contacting it with an elution buffer.
21. The method of embodiment 20 or 21, wherein the sample is tissue or tissue homogenate, cells, blood, plasma, serum, tissue fluid, CSF, bronchial fluid, mucous, semen, vaginal fluid, or amplified nucleic acids.
22. The method of embodiment 20, 21 or 22, wherein the solid-phase matrix that binds to a nucleic acid is contained in a spin column and wherein the nucleic acid bound to the solid-phase matrix is separated from unbound components of the digested sample by centrifugation; or wherein the solid-phase matrix comprises magnetic beads and wherein the magnetic beads are magnetically separated from unbound compounds of the digested sample.
23. The method of embodiment 20, 21, or 22, wherein the eluted nucleic acid is collected in a collection tube.
24. The method of embodiment 20, 21, 22, or 23, wherein the solid-phase matrix comprises silica particles.
25. The method of embodiment 20, 21, 22, 23 or 24, wherein the solid-phase matrix comprises silica particles bound to magnetic nanobeads that have a core size of no more than 30, 40, or 50 nm by transmission electron microscopy.
26. The method of embodiment 20, 21, 22, 23, 24, or 25, wherein the lysis buffer comprises Tris-HCl (pH 7), NaCl, guanidine thiocyanate, Na-EDTA, SDS and a buffer that adjusts a pH suitable for lysis or digestion of the sample to release nucleic acids.
27. The method of any one of embodiments 20-26, wherein the lysis buffer comprises 30 mM Tris-HCl (pH 7), 1M NaCl, 4.5M guanidine thiocyanate, 20 mM Na-EDTA, 0.5% w/v SDS-Buffer final pH 5.5, wherein the concentration of each ingredient may vary by +1, 2, 5, 10, 15 or 20%, and wherein the pH may vary from 5.0, 5.1, 5.2, 5.3, 5.4, 5.5, 5.6, 5.7, 5.8, to 6.0.
28. The method of any one of embodiments 20-27, wherein the binding buffer comprises an alcohol.
29. The method of any one of embodiments 20-28, wherein the binding buffer comprises 70, 80, 90-100% isopropyl alcohol.
30. The method of any one of embodiments 20-29, wherein the wash buffer comprises a first and second wash buffer,
   wherein the first wash buffer comprises: Tris-HCl, guanidine thiocyanate, ethanol; and
   wherein the second wash buffer comprises Tris-HCl (pH 7) and ethanol and is suitable for COL-NA; or comprises a dilute NaOH solution at pH 10.8 and is suitable for MG-NA, and
   wherein the first and second wash buffers are formulated so that they do not substantially remove nucleic acids from the solid-phase matrix.
31. The method of any one of embodiments 20-30, wherein the wash buffer comprises a first and second wash buffer,
   wherein the first wash buffer comprises 30 mM Tris-HCl (about pH 7), 1.125 M guanidine thiocyanate, 56% v/v ethanol and a buffer that adjusts pH to about 7.2 wherein the concentration of each ingredient in the first wash buffer may vary by +1, 2, 5, 10, 15 or 20%, and wherein the pH of the first wash buffer may vary from 6.13 to <9, and
   wherein the second wash buffer comprises 10 mM Tris-HCl (about pH 7), 70% v/v ethanol and a buffer that adjusts pH to about 6.3 to 8.3 suitable for COL-NA; or comprises a dilute NaOH solution at pH 10.8 and is suitable for MG-NA.
32. The method of any one of embodiments 20-31, wherein the elution buffer is water, a low salt buffer containing 10-50 mm monovalent salt ions, or a commercially available nucleic acid elution buffer.
33. The method of any one of embodiments 20-32, wherein the elution buffer is deionized water.
34. The method of any one of embodiments 20-33, wherein the lysis buffer or binding buffer does not contain linear polyacrylamide, glycogen, tRNA, sodium acetate, or another carrier or nucleic acid precipitant.
35. The method of any one of embodiments 20-34, wherein the lysis buffer or binding buffer further comprises linear polyacrylamide, glycogen, tRNA, sodium acetate, or another carrier or nucleic acid precipitant.
36. The method of any one of embodiments 20-35, wherein the sample comprises DNA.
37. The method of any one of embodiments 20-36, wherein the sample comprises RNA.
38. The method of any one of embodiments 20-37, wherein the sample comprises RNA and the lysis, binding, wash, and elution buffers do not contain RNAse.
39. The method of any one of embodiments 20-38, wherein the lysis buffer contains detergents at concentrations above their critical micelle concentration.
40. The method of any one of embodiments 20-39, wherein the binding buffer does not contain detergent.
41. The method of any one of embodiments 20-40, wherein the lysis buffer does not contain proteinase K or another protease.
42. The method of any one of embodiments 20-41, wherein the eluted nucleic acid has an A260/A280 ratio ranging from 1.8, 18.5, 1.9, 1.95 to 2.0.
43. The method of any one of embodiments 20-42, which yields the eluted nucleic acid in 10, 11, 12, 13, 14, 15, 16, 17, 18, 19 or 20 mins or less.
44. The method of any one of embodiments 20-43, wherein the eluted nucleic acid has an A260/A280 ratio ranging from 1.8, 18.5, 1.9, 1.95 to 2.0 which produces the eluted nucleic acid in 10, 11, 12, 13, 14, 15, 16, 17, 18, 19 or 20 mins or less.

### Background & Components

COL-NA and MG-NA methods and kits. The COL-NA and MG-NA kits provide a rapid 12-15 minutes protocol for recovering nucleic acids from a sample. These protocols were proved to efficiently recover nucleic acids isolation no matter the sample type. These rapid and simple protocols are compared to conventional commercial kits that require longer times to complete, such as kits provided by Abbott and Qiagen. Table 1 below compares extraction times required by commercially available kits demonstrating how much shorter in comparison the COL-NA and MG-NA protocols are.

TABLE 1
Length of extraction protocols for kits
offered by ThermoFischer Scientific

| | Method of | Extraction |
|---|---|---|
| Kit name | isolation | time (min) |
| PureLink RNA mini kit | Column based | 20 |
| Pure link RNA/DNA mini kit | | 45 |
| RNAqueous total RNA | | 25-30 |
| isolation kit | | |
| RecoverAll total nucleic | | 75 |
| acid isolation kit | | |
| MagMAX Cell-Free Total | Magnetic bead | 90 |
| Nucleic Acid Isolation Kit | based | |
| Dynabeads mRNA DIRECT | | 15 |
| Purification Kit* | | Note: |
| | | this kit is used for |
| | | mRNA isolation only |

Examples 2 and 3 further below describe specific differences between commercially available kits and extraction buffers and times.

Spin column. A silica particle-based spin column employs the binding affinity of DNA or RNA for silica particles immobilized in the column matrix of the spin column. The nucleic acid bind to the immobilized silica particles allowing unbound components to be washed away by centrifugal force. After washing the bound nucleic acid is eluted from the silica particles. In some embodiments, the silica matrix is composed of 4, 5, 6, 7, 8, or 9 layers of SiO₂ with average thickness 1-3 μm each. In one preferred embodiment spin columns having 6 layers are used. Typically, the silica particles bind to any nucleic acid but in some embodiments may be derivatized to preferentially bind to particular nucleic acid sequences. As mentioned above, such columns are commercially available and may be used in conjunction with the invention. In some embodiments, a spin column will contain silica particles, silica gel membrane, or an anion exchange resin which bind to nucleic acids.

The performance of the COL-NA and MG-NA buffers disclosed herein are the same without regard to the source of silica spin column. The results in FIGS. 10A and 10B show that when COL-NA buffers and the COL-NA protocol were applied in the extraction of SARS-CoV2 and HCV RNA however using silica spin columns from GENELUTE™ Total RNA Purification Kit (Sigma Aldrich), GeneAll, Qiagen or generic columns that are sold separately the same sample Ct was obtained. This confirms that extraction efficiency is attributed to the buffers and not to the silica column itself.

Spin columns may comprise a variety of varied sizes and types of silica that bind nucleic acids including silica gel, silica membrane such as a membrane of glass fiber matrix with attached or embedded silica particles, mesoporous silica, functionalized silica particles including those with added cationic groups that enhance nucleic acid binding, silica coated anion exchange resins, or beads coated with silica particles. However, other nucleic acid binding matrices are known and may be used instead of silica particles to bind nucleic acids such as anion-exchange resins, polymeric resins (which may be functionalized with moieties that bind nucleic acids), cellulose, glass fiber, graphene oxide, or hydroxyapatite. Other nucleic acid binding materials such as those described below for use with magnetic particles may be used.

A silica matrix based spin column is preferred for use with the invention as disclosed herein.

The same silica spin column was used in the isolation of HCV RNA ˜9.6 Kb in length, SARS-CoV2 RNA ˜29.9 kb, HBV DNA ˜3.2 kb. The same silica column was also used for mRNA isolation. FIG. 11 shows the results when COL-NA and GeneAll kits were used in the isolation of COL3 mouse mRNA from mouse liver homogenates and amplified by conventional PCR before being analyzed by gel electrophoresis. The thicker band observed with COL-NA indicated a higher extraction yield, see FIG. 11.

Magnetic nanoparticles or beads. Magnetic particle based separation of nucleic acids from a sample employ magnetic nanoparticles or beads coated with a nucleic acid binding material. This material selectively binds to the nucleic acids in the sample. After binding, the beads to which the nucleic acids are bound are magnetically separated. The separated beads may be washed to remove residual contaminants. Finally, an elution buffer is used to detach the nucleic acids from the magnetic particles.

Preferably, the magnetic nanoparticles or beads comprising or bound to a silica that binds to nucleic acids, such as those described above used in spin columns. Other non-silica materials including those described above for spin columns may comprise or be bound to magnetic or paramagnetic particles. Other nucleic acid binding materials may be used including amine-coated magnetic beads, carboxylated magnetic beads, paramagnetic beads which can be rapidly magnetized or demagnetizes, oligo-(dT) beads for binding poly-A tails of mRNAs, or streptavidin coated beads for binding to biotinylated nucleic acids. Magnetic beads may be synthesized by coprecipitation of ferric and ferrous chloride and then coated with tetraethyl orthosilicate (TEOS). The selection of specific ratios and amounts of magnetic bead substrates along with the composition of the storage buffer, such as MG-NA storage buffer described herein, produce smaller stable particles in the presence of a sample matrix in comparison to those described by Abbott. Further description of magnetic nanoparticles or beads is shown in FIG. 9.

In a preferred embodiment, MG-NA beads are silica coated magnetic nano-beads. More specifically they are magnetite particles synthesized by the co-precipitation of ferrous and ferric salts in different ratios. The particles preferably have a core size of 30, 40, or 50 nm and smaller by transmission electron microscopy. Core size may be measured by scanning electron microscopy (SEM), transmission electron microscopy (TEM) or atomic force microscopy (AFM).

Importantly they are stored in a specific storage buffer that allows their stabilization and reduces their aggregation in the presence of sample and lysis buffer components. In the presence of the aforementioned components the average hydrodynamic diameter 433±12 nm, unlike the beads from Abbott with are in micrometer range. In other embodiments the average hydrodynamic diameter may range from 50, 100, 150, 200, 250, 300, 350, 400, 450, 500, 550, 600, 650, 700, 750, 800, 850, 900, 950, or 1000 nm. HID may be measured or determined by dynamic light scattering (DLS) or nanoparticle tracking analysis (NTA).

Reme-D's nano-beads are usually suspended in a storage buffer composed of 287 mM NaCl, 2.7 mMKC1, 8 mM Na₂HPO₄, and 2 mM KH₂PO₄. A preferred MG-NA bead storage buffer comprises 287 mM NaCl, 2.7 mM KCl, 8 mM Na₂HPO₄, and 2 mM KH₂PO₄, wherein the content ranges may vary by 1, 2, 5, 10, 15 or 20%.

In preferred embodiments, Reme-D's beads are coated with tetraethoxyorthosilicate matrix (TEOS). However, in other embodiments, the beads may be coated with triethanolamine (TEA) or hexadecyltrimethylammonium p-toluenesulfonate (CTAB) or other materials; see <https://doi.org/10.1016/j.micromeso.2017.07.053> or cationic polymers such as chitosan, see DOI: 10.1039/DIAN01150B (both incorporated by reference).

Modified DNA or RNA includes chemically modified or chemically treated nucleic acids that comprise a negatively charged backbone under the conditions used in the COL-NA or MG-NA methods. Modified nucleic acids include xeno nucleic acids with modifications to their backbones, sugars, or nucleobases including nucleic acids that contain novel bases or base pairs. Nucleic acids and modified nucleic acids include those that are synthesized or recombinantly expressed.

Lysis Buffer. Proteinase K and other proteases. The preferred lysis buffer used in both the spin-column based method (COL-NA) and the magnetic bead based method (MG-NA) omits proteinase K and other proteases as well as other proteins.

The lysis buffer needs to break cells in sample and hence usually contains detergents. Detergents in lysis buffers are typically used above their critical micellar concentrations and hence will complex the DNA/RNA preventing their binding to the silica matrix; see Budker, V. G et al, Entrapment and condensation of DNA in neutral reverse micelles. BIOPHYSICAL JOURNAL 2002, 82 (3), 1570-1579. This DNA/RNA surfactant binding is enhanced for cationic and non-ionic surfactants and reduced with anionic surfactants, see Bhattacharya, S. & Mandal, S. S., Interaction of surfactants with DNA. Role of hydrophobicity and surface charge on intercalation and DNA melting. BIOCHIMICA ET BIOPHYSICA ACTA (BBA)-Biomembranes 1997, 1323 (1), 29-44.

The inventors considered determining whether selecting SDS for use in the lysis buffer might improve nucleic acid capture and yield and provide a more efficient method. The use of the SDS at the concentrations exemplified herein was also evaluated for its ability to inhibit RNAses in the sample. The inventors assessed whether the lysis buffer could effectively omit the use of RNAase inhibitors and proteinase K used in conventional kits/buffers; Bender, A. T, Enzymatic and chemical-based methods to inactivate endogenous blood ribonucleases for nucleic acid diagnostics. THE JOURNAL OF MOLECULAR DIAGNOSTICS 2020, 22 (8), 1030-1040.

In conjunction with the omission of proteinase K and RNAse inhibitors, the inventors increased the ionic strength of the buffer (e.g., to 1M NaCl) which was at least 10 fold higher than that conventionally used; Xia, Y.; et al., A modified SDS-based DNA extraction method from raw soybean. BIOSCIENCE REPORTS, 2019, 39 (2). In a preferred embodiment of the COL-NA or MG-NA methods, the lysis buffer comprises 30 mM Tris-HCl (pH 7), 1M NaCl, 4.5M guanidine thiocyanate, 20 mM Na-EDTA, 0.5% w/v SDS with a final pH of 5.5.

FIG. 4A shows images of a serum sample processed by the Qiagen method without proteinase K. FIG. 4B shows the serum sample processed using REME-D COL-NA proteinase K free buffer. As seen in the image, the sample without proteinase K treatment turns into a gel that could not be pipetted nor centrifuged through a silica column. When the same sample was processed using COL-NA lysis buffer without proteinase K a gel did not form (FIG. 4B). This demonstrates an advantage of the COL-NA method over conventional methods as proteinase K and other protein enzymes may be omitted avoiding a long incubation time that is needed for proteinase K to function, thus making the extraction protocol shorter. Also, the kits disclosed herein such as COL-NA and MG-NA avoid the need for refrigeration as proteinase K is typically stored at 4 or −20° C. The omission of proteinase K also reduces the cost of the kits.

Binding Buffer. The inventors typically omit from the binding buffer. They consider that detergents interfere with nucleic acid binding to the solid nucleic acid binding matrix. In a preferred embodiment, the COL-NA and MG-NA methods after lysis or digestion of a sample by the lysis buffer, a binding buffer containing about 100% isopropyl alcohol is applied. Preferably, the binding buffer used in the COL-NA or MG-NA methods does not include a carrier. This non-complex binding buffer does not contain other agents such as chaotropic salts or buffers such as Tris. In some embodiments, the lysis and binding buffers may be used together or combined.

In some non-preferred embodiments, a carrier may be included in a binding buffer. Carriers include carrier RNA, linearized polyacrylamide, glycogen or other carriers such as magnetic beads or gold nanoparticles.

Wash Buffer 1 is often employed to remove residual salts and contaminants from the sample after sample lysis or digestion. In some embodiments it includes a buffer such as Tris, EDTA or an alcohol such as ethanol. A preferred first wash buffer for the COL-NA or MG-NA comprises 30 mM Tris-HCl (pH 7), 1.125 M guanidine thiocyanate, 56% v/v ethanol-Buffer final pH 7.13.

Wash Buffer 2 is typically used to remove residual contaminants not removed by the first wash buffer. It may contain a higher concentration of alcohol like ethanol compared to the first wash buffer. A preferred second wash buffer for use with the COL-NA procedures comprises 10 mM Tris-HCl (pH 7) in 70% v/v ethanol-final pH 7.2, whereas for MG-NA procedures comprises diluted NaOH (pH 10.8).

Elution Buffer. This buffer is used to release nucleic acids bound the solid matrix. In many conventional methods it comprises a higher pH buffer to facilitate nucleic acid release. Elution buffers may contain sodium chloride, sodium phosphate, or imidazole. In a preferred embodiment of the COL-NA and MG-NA methods, the elution buffer is simply deionized water and/or nuclease-free water.

Universal buffers for DNA and RNA. A feature of the invention is that the same buffers may be used to isolated either RNA or DNA by the COL-NA or MG-NA methods. In contrast, kits from Abbott and Qiagen contain different buffers for DNA or RNA isolation indicating that their buffers do not perform in a comparable manner. This is also obvious from FIGS. 1A-1C and FIGS. 6A-6C where Abbott DNA kits did not perform as well as their RNA kit in the extraction of HCV & SARS-CoV2 RNA. For comparison, the Reme-D and Abbott buffers are described below.

For COL-NA and MG-NA the buffers described in Table 2 below were used to generate all the results reported. The commercial kits were used according the kit's instructions.

TABLE 2

| | Reme-D | Abbott (1) |
|---|---|---|
| Lysis buffer | 30 mM Tris-Hydrogen | 100 mM Tris solution |
| | chloride | containing Guanidinium |
| | 1M Sodium chloride | thiocyanate and |
| | 4.5M Guanidium thiocyanate | detergent. |
| | 20 mM EDTA sodium salt | |
| | 0.5% Sodium dodecyl | |
| | sulphate | |
| | (0.1% Beta-mercaptoethanol | |
| | for MG-NA only) | |
| Binding buffer | Isopropanol | |
| Wash 1 | 30 mM Tris-Hydrogen | 50 mM Acetate solution |
| | chloride | containing Guanidinium |
| | 1.125M Guanidium | thiocyanate and |
| | thiocyanate | detergent |
| | 56% Ethanol | |
| Wash 2 | 10 mM Tris-Hydrogen | Nuclease free water |
| | chloride in70% Ethanol | |
| | (For COL-NA) | |
| | Diluted NaOH solution- pH | |
| | 10.8 (For MG-NA) | |
| Elution buffer | De-ionized water | |
| Magnetic beads | 50 mg/ml magnetic | 1.5% microparticles in |
| | nanoparticles in | 50% lysis buffer |
| | 287 mM sodium chloride, | |
| | 2.7 mM potassium chloride, | |
| | 8 mM disodium hydrogen | |
| | phosphate, and | |
| | 2 mM potassium dihydrogen | |
| | phosphate | |

In Table 2, MG-NA wash 2 was diluted NaOH (pH 10.8), which was prepared by dissolving a given amount of NaOH in water. COL-NA wash 2 was 10 mM Tris-Hydrogen chloride in 70% Ethanol.

### Example 1

### Preferred Buffers for RNA and DNA Recovery

In one preferred but non-limiting embodiment of the COL-NA or MG-NA methods, the following buffers are used.

MG-NA storage buffer: The magnetic beads are stored which is made of 287 mM sodium chloride, 2.7 mM potassium chloride, 8 mM disodium hydrogen phosphate, and 2 mM potassium dihydrogen phosphate.

COL-NA Lysis buffer: 30 mM Tris-HCl (pH 7), 1M NaCl, 4.5M guanidine thiocyanate, 20 mM Na-EDTA, 0.5% w/v SDS-Buffer final pH 5.5.

MG-NA lysis buffer: 30 mM Tris-HCl (pH 7), 1M NaCl, 4.5M guanidine thiocyanate, 20 mM Na-EDTA, 0.5% w/v SDS-Buffer final pH 5.5 with added beta-mercaptoethanol.

Binding buffer: 100% iso-propanol.

Wash 1: 30 mM Tris-HCl (pH 7), 1.125 M guanidine thiocyanate, 56% v/v ethanol-Buffer final pH 7.13.

Wash 2 COL-NA: 10 mM Tris-HCl (pH 7) in 70% v/v ethanol-Final pH 7.2.

Wash 2 MG-NA: diluted NaOH solution, pH 10.8.

Elution buffer: De-ionized water.

### Example 2

### COL-NA Method

The COL-NA method was used to extract HBV DNA, HCV RNA and SARS-COV-2 RNA from samples. The HCV and Hepatitis B samples were serum and the SARS-COV-2 sample was from a nasopharyngeal swab.

The COL-NA kit is universal and extracts nucleic acids from a variety of different samples. For example, these include from the serum samples and nasopharyngeal samples mentioned above as well as from other samples, such as murine liver homogenates as shown in FIG. 12. For both COL-NA and MG-NA, the buffers described in Table 2 above were used to generate all the results reported. The commercial kits were also used according the kit's instructions.

The COL-NA method was performed using the steps described by FIG. 2A.

In parallel, using the same samples, the conventional QIamp DNA Minikit, QIAamp Mini Elute Virus Spin and GeneAll methods were performed using the recommended steps shown by FIGS. 2B and 2C.

The results of these methods are shown in FIGS. 1A-IC. All samples were run in duplicate. As shown by FIG. 1A, the COL-NA method provided superior recovery of HBV DNA compared to QIAmp and GeneAll methods are comparable recovery to QIAamp Minielute. However as apparent from FIGS. 2A-2C, the COL-NA method used fewer steps, simpler buffer formulations, and was faster being complete within 15 mins while QIAamp DNA Mini Kit took 20 minutes.

As shown by FIG. 1B, the COL-NA method provided superior recovery of HCV RNA compared to QIAmp and GeneAll methods are somewhat less recovery than the QIAamp Minielute. However, as noted above, the COL-NA method was complete within 15 mins while the QIAamp Minielute took 20 mins and used more complex buffers such as lysis buffers containing PK.

As shown by FIG. 1C, the COL-NA method provided superior recovery of SARS-COV-2 RNA compared to both QIAmp methods and compared to the GeneAll methods. The COL-NA method was complete in 15 mins, while the QIAamp Minielute took 33 mins, the QIAamp Minikit took 20 mins and used more complex buffers, such as lysis buffers containing PK.

As shown in FIG. 3A-3C, the addition of a commercial Poly-A carrier purchased from Qiagen did not substantially affect recovery of viral DNA or RNA form HCV, HBV or SARS-CoV-2. This shows that the COL-NA method may be performed without the use of a carrier thus simplifying it compared to commercial kits which require a carrier to enhance nucleic acid recovery. The lysis/binding buffer used here makes the use of carrier unnecessary. FIGS. 3A-3C show extraction yield obtained with COL-NA with and without carrier showing that performance is not affected.

As shown by FIGS. 4A and 4, proteinase K was essential to obtain liquid samples from a serum sample using the Qiagen kit. The COL-NA method does not require the use of PK to isolated nucleic acids from serum and does not produce a gelled sample.

As shown by FIG. 5, the extraction efficiency of HCV RNA using the two Qiagen kits was decreased when the 10 min incubation step was omitted. In contrast, the COL-NA method, which does not contain proteinase K and thus does not need to be incubated for the proteinase K to work, omits this step and obtains superior extraction efficiency in 15 mins total.

### Example 3

### MG-NA Method

The MG-NA method was used to extract HBV DNA, HCV RNA and SARS-COV-2 RNA from samples. HCV and HBV containing serum and SARS-COV-2 containing nasopharyngeal swab samples were used. Like COL-NA, MG-NA is a universal nucleic acid extraction kit. It can be used for extraction of DNA/RNA from a variety of diverse types of samples such cell culture (data not shown) and tissue homogenates as shown by FIG. 12, which shows 4 replicas in which COL3 mRNA was extracted from mouse liver homogenates. The extracted mRNA was then amplified and analyzed by gel electrophoresis.

For COL-NA and MG-NA, the buffers described in the Table above were used to generate all the results reported. The commercial kits were used according the kit's instructions.

The MG-NA method was performed using the steps described by FIG. 7A.

In parallel, using the same samples, the conventional Abbott mSample Preparation System and Abbott mSample Preparation System DNA methods were performed using the recommended steps shown by FIGS. 7B and 7C.

The results of these methods are shown in FIGS. 6A-6C. Each sample was run in duplicate, for example, FIG. 6A for Abbott-RNA shows the results—32 and 32.2—from duplicate samples.

As shown by FIG. 6A, the MG-NA method provided approximately equivalent recovery HBV DNA compared to the two Abbott kits. However as apparent from FIGS. 7A-7C, the MG-NA method used fewer steps, simpler buffer formulations, and was faster being complete within 12 mins while the two Abbott kits took 44 or 87 mins.

As shown by FIG. 6B, the MG-NA method provided equivalent recovery of HCV RNA compared to the two Abbott kits. The results obtained by MG-NA were similar to those from the Abbott RNA kit but superior to those of the Abbott DNA kit. However as apparent from FIGS. 7A-7C, the MG-NA method used fewer steps, simpler buffer formulations, and was faster being complete within 12 mins while the two Abbott kits took 44 or 87 mins.

As shown by FIG. 6C, the MG-NA method provided superior recovery of SARS-COV-2 RNA compared to the Abbott DNA kit and GeneAll kit and comparable recovery to the Abbott RNA kit. However as apparent from FIGS. 7A-7C, the MG-NA method used fewer steps, simpler buffer formulations, and was faster being complete within 12 mins while the two Abbott kits took 44 or 87 mins.

Terminology. Terminology used herein is for the purpose of describing particular embodiments only and is not intended to be limiting of the invention.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology. As referred to herein, all compositional percentages are by weight of the total composition, unless otherwise specified.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

1. A nucleic acid extraction kit comprising a solid-phase matrix that binds nucleic acid, a lysis buffer, a binding buffer, a wash buffers, and an elution buffer;
wherein the solid-phase matrix comprises nucleic acid binding silica particles and wherein the solid phase matrix is contained within a spin column or is bound to magnetic nanoparticles;
wherein the lysis buffer comprises Tris-HCl, NaCl, guanidine thiocyanate, Na-EDTA and has a pH suitable for lysis or digestion of the sample and release of nucleic acids;
wherein the binding buffer comprises isopropyl alcohol,
wherein the wash buffer(s) comprise a first wash buffer comprising Tris-HCl, guanidine thiocyanate, ethanol; and a second wash buffer comprising Tris-HCl and ethanol when the kit contains a solid phase matrix contained within a spin column or comprises a diluted NaOH solution at pH 10.8 when the solid phase matrix is bound to magnetic particles.

2. The nucleic acid extraction kit of claim 1, wherein the solid phase matrix comprises silica particles that bind to nucleic acids.

3. The nucleic acid extraction kit of claim 1, further comprising a collection rack and/or at least one collection tube.

4. The nucleic acid extraction kit of claim 1, wherein the solid phase matrix is contained in a spin column.

5. The nucleic acid extraction kit of claim 1, further comprising a collection rack and/or at least one collection tube.

6. The nucleic acid extraction kit of claim 1, wherein the binding buffer comprises 70-100% v/v isopropanol.

7. The nucleic acid extraction kit of claim 1, wherein the solid phase matrix is contained within a spin column and wherein the lysis buffer comprises 30 mM Tris-HCl (pH 7), 1M NaCl, 4.5M guanidine thiocyanate, 20 mM Na-EDTA, 0.5% w/v SDS at a final pH of 5.5, wherein the concentration of each ingredient may vary by +1, 2, 5, 10, 15 or 20%, and wherein the pH may vary from 5.0, 5.1, 5.2, 5.3, 5.4, 5.5, 5.6, 5.7, 5.8, to 6.0.

8. The nucleic acid extraction kit of claim 1, wherein the solid phase matrix is bound to magnetic nanoparticles.

9. The nucleic acid extraction kit of claim 1, wherein the solid phase matrix is bound to magnetic particles that are magnetic nanobeads having a core size of no more than 30, 40, or 50 nm by transmission electron microscopy, wherein the lysis buffer comprises 30 mM Tris-HCl (pH 7), 1M NaCl, 4.5M guanidine thiocyanate, 20 mM Na-EDTA, 0.5% w/v SDS, and 0.1 v/v % β-mercaptoethanol at a final pH of 5.5, wherein the concentration of each ingredient may vary by ±1, 2, 5, 10, 15 or 20%, and wherein the pH may vary from 5.0, 5.1, 5.2, 5.3, 5.4, 5.5, 5.6, 5.7, 5.8, to 6.0

10. The nucleic acid extraction kit of claim 1, wherein the elution buffer is water or a low salt buffer containing 10-50 mm monovalent salt ions.

11. A method for extracting a nucleic acid from a sample comprising:
contacting the sample with a lysis buffer for a time and under conditions suitable to release the nucleic acid from other components in the sample,
contacting the released nucleic acid with a binding buffer to facilitate binding of the nucleic acid with a solid phase matrix in a spin column,
passing the nucleic acid through the spin column for a time and under conditions suitable for binding of the released nucleic acid to the solid phase matrix,
washing the solid phase matrix that is bound to the released nucleic acid one or more times to remove unbound components in the sample,
contacting the washed solid phase matrix bound to the nucleic acid with an elution buffer under conditions suitable for release of the bound nucleic acid from the solid phase matrix,
thereby extracting the nucleic acid from the sample.

12. The method of claim 11, wherein the sample is tissue or tissue homogenate, cells, blood, plasma, serum, tissue fluid, CSF, bronchial fluid, mucous, semen, vaginal fluid, or amplified nucleic acids.

13. The method of claim 11, wherein the lysis buffer comprises Tris-HCl, NaCl, guanidine thiocyanate, Na-EDTA and has a pH suitable for lysis or digestion of the sample and release of nucleic acids and wherein the binding buffer comprises 70-100% v/v isopropyl alcohol.

14. The method of claim 11, wherein said washing comprises contacting the solid phase matrix bound to the nucleic acid with a first and second wash buffer, wherein the first wash buffer comprises Tris-HCl, guanidine thiocyanate, and ethanol; and wherein the second wash buffer comprises Tris-HCl and ethanol.

15. The method of claim 11, wherein said elution buffer is water, a low salt buffer containing 10-50 mm monovalent salt ions.

16. A method for extracting a nucleic acid from a sample comprising:
contacting the sample with a lysis buffer for a time and under conditions suitable to release the nucleic acid from other components in the sample,
contacting the released nucleic acid with a binding buffer to facilitate binding of the nucleic acid with a solid phase matrix that binds to nucleic acids bound to magnetic nanoparticles.
contacting the nucleic acid with the magnetic nanoparticles for a time and under conditions suitable for binding of the nucleic acid to the solid phase matrix bound to magnetic nanoparticles,
magnetically separating the nucleic acid bound to the solid phase matrix bound to magnetic nanoparticles,
washing the solid phase matrix bound to magnetic nanoparticles which is bound to the released nucleic acid one or more times to remove unbound components in the sample,
contacting the solid phase matrix bound to magnetic nanoparticles which is bound to the released nucleic acid with an elution buffer under conditions suitable for release of the bound nucleic acid from the solid phase matrix, thereby extracting the nucleic acid from the sample.

17. The method of claim 16, wherein the sample is tissue or tissue homogenate, cells, blood, plasma, serum, tissue fluid, CSF, bronchial fluid, mucous, semen, vaginal fluid, or amplified nucleic acids.

18. The method of claim 16, wherein the lysis buffer comprises wherein the lysis buffer comprises Tris-HCl (pH 7), NaCl, guanidine thiocyanate, Na-EDTA, SDS, β-mercaptoethanol and wherein the binding buffer comprises 70-100% v/v isopropyl alcohol.

19. The method of claim 16, wherein said washing comprises contacting the solid phase matrix bound to the nucleic acid with a first and second wash buffer, wherein the first wash buffer comprises Tris-HCl, guanidine thiocyanate, ethanol; and wherein the second wash buffer comprises a diluted NaOH solution at pH 10.8.

20. The method of claim 16, wherein said elution buffer is water, a low salt buffer containing 10-50 mm monovalent salt ions.
